(19) United States
(12) Patent Application Publication
(10) Pub. No.: US 2020/0054628 A1
(43) Pub. Date: Feb. 20, 2020

(54) Therapeutic Polymeric Nanoparticles Comprising Lipids and Methods of Making and Using Same

(71) Applicant: Pfizer Inc., New York, NY (US)

(72) Inventor: Young-Ho Song, Natick, MA (US)

(73) Assignee: Pfizer Inc., New York, NY (US)

(21) Appl. No.: 16/343,009

(22) PCT Filed: Oct. 17, 2017
(86) PCT No.: PCT/IB2017/056439

§ 371 Date: Apr. 18, 2019

Related U.S. Application Data
(60) Provisional application No. 62/410,551, filed on Oct. 20, 2016.

(51) Int. Cl. A61K 31/4965; A61K 31/337; A61K 47/34; A61K 47/10; A61K 9/14

(52) U.S. Cl. CPC A61K 31/4965; A61K 31/337; A61K 9/14; A61K 47/10; A61K 47/34

(57) ABSTRACT

The present disclosure generally relates to therapeutic nanoparticles. Exemplary nanoparticles disclosed herein may include about 10 to about 70 weight percent of biocompatible polymers such as a di-block polymer (for example, poly(lactic)acid and polyethylene glycol or poly(lactic)-co-poly (glycolic) acid and poly(ethylene)glycol), about 5 to about 50 weight percent glyceride (for example, a monoglyceride, a diglyceride, or a triglyceride), and about 0.1% to about 40% weight percent therapeutic agent (for example, docetaxel or bortezomib).

### BACKGROUND

Systems that deliver certain drugs to a patient (e.g., targeted to a particular tissue or cell type or targeted to a specific diseased tissue but not normal tissue), or that control release of drugs has long been recognized as beneficial. For example, therapeutics that include an active drug and that are capable of locating in a particular tissue or cell type, e.g., a specific diseased tissue, may reduce the amount of the drug in tissues of the body that do not require treatment. This is particularly important when treating a condition such as cancer where it is desirable that a cytotoxic dose of the drug is delivered to cancer cells without killing the surrounding non-cancerous tissue. Further, such therapeutics may reduce the undesirable and sometimes life- threatening side effects common in anticancer therapy. For example, nanoparticle therapeutics may, due to the small size, evade recognition within the body allowing for targeted and controlled delivery while, e.g., remaining stable for an effective amount of time.

Therapeutics that offer such therapy and/or controlled release and/or targeted therapy also must be able to deliver an effective amount of drug. It can be a challenge to prepare nanoparticle systems that have an appropriate amount of drug associated each nanoparticle, while keeping the size of the nanoparticles small enough to have advantageous delivery properties. For example, while it is desirable to load a nanoparticle with a high quantity of therapeutic agent, nanoparticle preparations that use a drug load that is too high will result in nanoparticles that are too large for practical therapeutic use. Further, it may be desirable for therapeutic nanoparticles to remain stable so as to, e.g., substantially limit rapid or immediate release of the therapeutic agent.

Accordingly, a need exists for new nanoparticle formulations and methods of making such nanoparticles and compositions, that can deliver therapeutic levels of drugs to treat diseases such as cancer, while also reducing patient side effects.

### SUMMARY

In one aspect, the invention provides a therapeutic nanoparticle that includes a therapeutic agent, e.g. taxane or bortezomib, one, two, or three biodegradable polymers, and a glyceride. For example, disclosed herein is a therapeutic nanoparticle comprising about 0.1 to about 40 weight percent of a therapeutic agent; about 10 to about 70 weight percent polymer (e.g. a diblock copolymer of poly(lactic) acid and polyethylene (glycol) or a diblock copolymer of poly(lactic)-co-poly (glycolic) acid-poly(ethylene)glycol); and about 5 to about 50 weight percent glyceride (e.g. a monoglyceride, a diglyceride, or a triglyceride). In an embodiment, the glyceride is a monoglyceride. In another embodiment the monoglyceride is lauroyl-rac-glycerol. The glyceride may be homogeneously dispersed within the nanoparticle. Exemplary therapeutic agents include, but are not limited to, antineoplastic agents such as taxanes, e.g. docetaxel or a boronate compound, e.g. bortezomib.

In an exemplary embodiment, the therapeutic nanoparticle may include about 30% to about 40% by weight PLA-PEG block copolymer (e.g., PEG (5,000 Da)/PLA (16,000 Da)), about 10% to about 40% by weight glyceride, or about 20 to about 50 weight percent glyceride, and about 20% to about 40% by weight boronate compound such as bortezomib. In another embodiment, the particles may include about 30% to about 40% by weight PLA-PEG block copolymer (e.g., PEG (5,000 Da)/PLA (50,000 Da)), about 10% to about 40% by weight glyceride, or about 20 to about 50 weight percent glyceride, and about 20% to about 40% by weight boronate compound such as bortezomib.

In yet another embodiment, a biocompatible, therapeutic polymeric nanoparticle contemplated herein may include a taxane (for example, docetaxel). In an exemplary embodiment, the particles may include about 50% to about 70% by weight PLA-PEG block copolymer (e.g., PEG (5,000 Da)/PLA (16,000 Da)), about 5% to about 40% by weight glyceride, or about 10 to about 30 by weight glyceride, and about 20% to about 40% by weight a taxane such as docetaxel. In another embodiment, the particles may include about 30% to about 40% by weight PLA-PEG block copolymer (e.g., PEG (5,000 Da)/PLA (50,000 Da)), about 30% to about 40% by weight glyceride, and about 20% to about 40% by weight a taxane such as docetaxel.

Compositions are provided such as compositions comprising a plurality of disclosed nanoparticles and a pharmaceutically acceptable excipient.

Also contemplated herein are methods of making disclosed nanoparticles and methods of treating cancers and/or other indications such as multiple myeloma comprising administering to a patient in need thereof a disclosed particle or composition.

In another embodiment, provided herein is plurality of therapeutic nanoparticles prepared by combining a therapeutic agent (e.g. docetaxel or bortezomib), a diblock poly(lactic)acid-polyethylene glycol or poly(lactic)-co-poly (glycolic) acid-poly(ethylene)glycol, and a glyceride (a monoglyceride, a diglyceride, or a triglyceride) with an organic solvent to form a first organic phase having about 10 to about 40% solids; combining the first organic phase with a first aqueous solution to form a second phase; emulsifying the second phase to form an emulsion phase; quenching the emulsion phase to form a quenched phase; adding a drug solubilizer to the quenched phase to form a solubilized phase of unencapsulated therapeutic agent; and filtering the solubilized phase to recover the nanoparticles, thereby forming a slurry of therapeutic nanoparticles each having about 0.1 to about 40 weight percent of the therapeutic agent. In an embodiment, the glyceride is a monoglyceride. In another embodiment the monoglyceride is lauroyl-rac-glycerol. The glyceride may be homogeneously dispersed within the nanoparticle.

### BRIEF DESCRIPTION OF THE DRAWINGS

FIG. 1 is a flow chart for an emulsion process for forming disclosed nanoparticles.

FIG. 2 is a flow diagram for a disclosed emulsion process.

FIG. 3A-B depict in vitro release of bortezomib of various nanoparticles disclosed herein.

FIG. 4 depicts in vitro release of docetaxel of various nanoparticles disclosed herein.

### DETAILED DESCRIPTION

The present invention generally relates to polymeric nanoparticles that include an active or therapeutic agent or drug, and methods of making and using such therapeutic nanoparticles. In general, a “nanoparticle” refers to any particle having a diameter of less than 1000 nm, e.g. about 10 nm to about 200 nm. Disclosed therapeutic nanoparticles may include nanoparticles having a diameter of about 60 to about 190 nm, or about 70 to about 190 nm, or about 60 to about 180 nm, about 70 nm to about 180 nm, or about 50 nm to about 200 nm.

Disclosed nanoparticles may include about 0.1 to about 40 weight percent, about 0.1 to about 30 weight percent, about 0.1 to about 20 weight percent, or about 1 to about 30 weight percent of a therapeutic agent, such as an antineoplastic agent, e.g. a taxane agent (for example, docetaxel) or a peptide boronic acid compound (for example, bortezomib)

Nanoparticles disclosed herein include one, two, three or more biocompatible and/or biodegradable polymers and a glyceride. For example, a contemplated nanoparticle may include about 10 to about 70 weight percent of biocompatible polymers such as a diblock polymer (for example, poly(lactic)acid and polyethylene glycol or poly(lactic)-co-poly (glycolic) acid and poly(ethylene)glycol), about 5 to about 50 weight percent glyceride (e.g. a monoglyceride, a diglyceride, or a triglyceride), and about 0.1 to about 40 weight percent of a therapeutic agent (for example, docetaxel or bortezomib).

The features and other details of the disclosure will now be more particularly described. Before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

### Definitions

The term “glycerides” as used herein refers to esters formed from glycerol and fatty acids. Glycerol has three hydroxyl functional groups, which can be esterified with one, two, or three fatty acids. Glycerides can be monoglycerides, diglycerides, and triglycerides.

The term “monoglycerol lipid” or “monoglyceride” as used herein refers to a glyceride consisting of one fatty acid chain covalently bonded to a glycerol molecule through an ester linkage. Monoglycerol lipid can be broadly divided into two groups: 1-monoacylglycerols and 2-monoacylglycerols, depending on the position of the ester bond on the glycerol moiety. Exemplary monoglycerol lipids include, but are not limited to, lauroyl-rac-glycerol, glycerol monomyristate, glycerol monopalmitate, glycerol monostearate, glycerol monoarachidate, glycerol monobehenate, glycerol monopalmitoleate, glycerol monooleate, glycerol monolinoleate, glycerol monolinolenate, glycerol monoarachidonate, and glycerol monocaprylate, and/or for example 1-monomyristoyl-rac glycerol, 1-mono-palmitoyl-rac-glycerol, 2-monopalmitoylglycerol, 1-mono-palmitolenyl-rac-glycerol, 1-monostearoyl-rac-glycerol, 1-monoleoyl-rac-glycerol, 1-monolinoleoyl-rac-glycerol, and 1-monolinolenoyl-rac-glycerol or combinations thereof.

The term “diglyceride” as used herein refers to a glyceride consisting of two fatty acid chain covalently bonded to a glycerol molecule through an ester linkage.

Exemplary diglycerides include, but are not limited to, glycerol dilaurate, glycerol dimyristate, glycerol dipalmitate, glycerol distearate, glycerol diarachidate, glycerol dibehenate, glycerol dipalmitoleate, glycerl dioleate, glycerol dilinoleate, glycerol dilinolenate, glycerol diarachidonate, or combinations thereof.

The term “triglyceride” as used here refers to a glyceride consisting of three fatty acid chain covalently bonded to a glycerol molecule through an ester linkage. Exemplary diglycerides include, but are not limited to, glycerol trilaurate, glycerol trimyristate, glycerol tripalmitate, glycerol tristearate, glycerol triarachidate, glycerol tribehenate, glycerol tripalmitoleate, glycerl trioleate, glycerol trilinoleate, glycerol trilinolenate, glycerol triarachidonate, or combinations thereof.

“Treating” includes any effect, e.g., lessening, reducing, modulating, or eliminating, that results in the improvement of the condition, disease, disorder and the like.

“Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

“Individual,” “patient,” or “subject” are used interchangeably and include any animal, including mammals, such as mice, rats, other rodents, rabbits, dogs, cats, swine, cattle, sheep, horses, or primates, and most preferably humans. The compounds and compositions of the invention can be administered to a mammal, such as a human, but can also be other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). “Modulation” includes antagonism (e.g., inhibition), agonism, partial antagonism and/or partial agonism.

In the present specification, the term “therapeutically effective amount” means the amount of the subject compound or composition that will elicit the biological or medical response of a tissue, system, animal or human that is being sought by the researcher, veterinarian, medical doctor or other clinician. The compounds and compositions of the invention are administered in therapeutically effective amounts to treat a disease. Alternatively, a therapeutically effective amount of a compound is the quantity required to achieve a desired therapeutic and/or prophylactic effect.

The term “pharmaceutically acceptable salt(s)” as used herein refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions that are acidic in nature are capable of forming base salts with various pharmacologically acceptable cations. Examples of such salts include alkali metal or alkaline earth metal salts, such as calcium, magnesium, sodium, lithium, zinc, potassium, and iron salts.

### Therapeutic Particles

Contemplated biocompatible, therapeutic polymeric nanoparticles include a therapeutic agent, a biodegradable polymer and/or biocompatible polymer, and a glyceride.

In some embodiments, disclosed nanoparticles include a matrix of polymers. Disclosed nanoparticles may include one or more polymers, e.g. a diblock co-polymer and/or a monopolymer. Disclosed therapeutic nanoparticles include a therapeutic agent that can be associated with the surface of, encapsulated within, surrounded by, and/or dispersed throughout a polymeric matrix.

A wide variety of polymers and methods for forming particles therefrom are known in the art of drug delivery. In some embodiments, the disclosure is directed toward nanoparticles with at least one polymer, for example, a first polymer that may be a co-polymer, e.g. a diblock co-polymer, and optionally a polymer that may be for example a homopolymer.

Any polymer can be used in accordance with the present invention. Polymers can be natural or unnatural (synthetic) polymers. Polymers can be homopolymers or copolymers comprising two or more monomers. In terms of sequence, copolymers can be random, block, or comprise a combination of random and block sequences. Contemplated polymers may be biocompatible and/or biodegradable.

The term “polymer,” as used herein, is given its ordinary meaning as used in the art, i.e., a molecular structure comprising one or more repeat units (monomers), connected by covalent bonds. The repeat units may all be identical, or in some cases, there may be more than one type of repeat unit present within the polymer. In some cases, the polymer can be biologically derived, i.e., a biopolymer. Non-limiting examples include peptides or proteins. In some cases, additional moieties may also be present in the polymer, for example biological moieties such as those described below. If more than one type of repeat unit is present within the polymer, then the polymer is said to be a “copolymer.” It is to be understood that in any embodiment employing a polymer, the polymer being employed may be a copolymer in some cases. The repeat units forming the copolymer may be arranged in any fashion. For example, the repeat units may be arranged in a random order, in an alternating order, or as a block copolymer, i.e., comprising one or more regions each comprising a first repeat unit (e.g., a first block), and one or more regions each comprising a second repeat unit (e.g., a second block), etc. Block copolymers may have two (a diblock copolymer), three (a triblock copolymer), or more numbers of distinct blocks.

Disclosed particles can include copolymers, which, in some embodiments, describes two or more polymers (such as those described herein) that have been associated with each other, usually by covalent bonding of the two or more polymers together. Thus, a copolymer may comprise a first polymer and a second polymer, which have been conjugated together to form a block copolymer where the first polymer can be a first block of the block copolymer and the second polymer can be a second block of the block copolymer. Of course, those of ordinary skill in the art will understand that a block copolymer may, in some cases, contain multiple blocks of polymer, and that a “block copolymer,” as used herein, is not limited to only block copolymers having only a single first block and a single second block. For instance, a block copolymer may comprise a first block comprising a first polymer, a second block comprising a second polymer, and a third block comprising a third polymer or the first polymer, etc. In some cases, block copolymers can contain any number of first blocks of a first polymer and second blocks of a second polymer (and in certain cases, third blocks, fourth blocks, etc.). In addition, it should be noted that block copolymers can also be formed, in some instances, from other block copolymers. For example, a first block copolymer may be conjugated to another polymer (which may be a homopolymer, a biopolymer, another block copolymer, etc.), to form a new block copolymer containing multiple types of blocks, and/or to other moieties (e.g., to non-polymeric moieties).

In some embodiments, the polymer (e.g., copolymer, e.g., block copolymer) can be amphiphilic, i.e., having a hydrophilic portion and a hydrophobic portion, or a relatively hydrophilic portion and a relatively hydrophobic portion. A hydrophilic polymer can be one generally that attracts water and a hydrophobic polymer can be one that generally repels water. A hydrophilic or a hydrophobic polymer can be identified, for example, by preparing a sample of the polymer and measuring its contact angle with water (typically, the polymer will have a contact angle of less than 60°, while a hydrophobic polymer will have a contact angle of greater than about 60°). In some cases, the hydrophilicity of two or more polymers may be measured relative to each other, i.e., a first polymer may be more hydrophilic than a second polymer. For instance, the first polymer may have a smaller contact angle than the second polymer.

In one set of embodiments, a polymer (e.g., copolymer, e.g., block copolymer) contemplated herein includes a biocompatible polymer, i.e., the polymer that does not typically induce an adverse response when inserted or injected into a living subject, for example, without significant inflammation and/or acute rejection of the polymer by the immune system, for instance, via a T-cell response. Accordingly, the therapeutic particles contemplated herein can be non-immunogenic. The term non-immunogenic as used herein refers to endogenous growth factor in its native state which normally elicits no, or only minimal levels of, circulating antibodies, T-cells, or reactive immune cells, and which normally does not elicit in the individual an immune response against itself.

Biocompatibility typically refers to the acute rejection of material by at least a portion of the immune system, i.e., a nonbiocompatible material implanted into a subject provokes an immune response in the subject that can be severe enough such that the rejection of the material by the immune system cannot be adequately controlled, and often is of a degree such that the material must be removed from the subject. One simple test to determine biocompatibility can be to expose a polymer to cells in vitro; biocompatible polymers are polymers that typically will not result in significant cell death at moderate concentrations, e.g., at concentrations of 50 micrograms/10⁶ cells. For instance, a biocompatible polymer may cause less than about 20% cell death when exposed to cells such as fibroblasts or epithelial cells, even if phagocytosed or otherwise uptaken by such cells. Non-limiting examples of biocompatible polymers that may be useful in various embodiments of the present invention include polydioxanone (PDO), polyhydroxyalkanoate, polyhydroxybutyrate, poly(glycerol sebacate), polyglycolide, polylactide, PLGA, polycaprolactone, or copolymers or derivatives including these and/or other polymers.

In certain embodiments, contemplated biocompatible polymers may be biodegradable, i.e., the polymer is able to degrade, chemically and/or biologically, within a physiological environment, such as within the body. As used herein, “biodegradable” polymers are those that, when introduced into cells, are broken down by the cellular machinery (biologically degradable) and/or by a chemical process, such as hydrolysis, (chemically degradable) into components that the cells can either reuse or dispose of without significant toxic effect on the cells. In one embodiment, the biodegradable polymer and their degradation byproducts can be biocompatible.

For instance, a contemplated polymer may be one that hydrolyzes spontaneously upon exposure to water (e.g., within a subject), the polymer may degrade upon exposure to heat (e.g., at temperatures of about 37° C.). Degradation of a polymer may occur at varying rates, depending on the polymer or copolymer used. For example, the half-life of the polymer (the time at which 50% of the polymer can be degraded into monomers and/or other nonpolymeric moieties) may be on the order of days, weeks, months, or years, depending on the polymer. The polymers may be biologically degraded, e.g., by enzymatic activity or cellular machinery, in some cases, for example, through exposure to a lysozyme (e.g., having relatively low pH). In some cases, the polymers may be broken down into monomers and/or other nonpolymeric moieties that cells can either reuse or dispose of without significant toxic effect on the cells (for example, polylactide may be hydrolyzed to form lactic acid, polyglycolide may be hydrolyzed to form glycolic acid, etc.).

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids or polyanhydrides.

In other embodiments, contemplated polyesters for use in disclosed nanoparticles may be diblock copolymers, e.g., PEGylated polymers and copolymers (containing poly(ethylene glycol) repeat units) such as of lactide and glycolide (e.g., PEGylated PLA, PEGylated PGA, PEGylated PLGA), PEGylated poly(caprolactone), and derivatives thereof. For example, a “PEGylated” polymer may assist in the control of inflammation and/or immunogenicity (i.e., the ability to provoke an immune response) and/or lower the rate of clearance from the circulatory system via the reticuloendothelial system (RES), due to the presence of the poly(ethylene glycol) groups.

PEGylation may also be used, in some cases, to decrease charge interaction between a polymer and a biological moiety, e.g., by creating a hydrophilic layer on the surface of the polymer, which may shield the polymer from interacting with the biological moiety. In some cases, the addition of poly(ethylene glycol) repeat units may increase plasma half-life of the polymer (e.g., copolymer, e.g., block copolymer), for instance, by decreasing the uptake of the polymer by the phagocytic system while decreasing transfection/uptake efficiency by cells. Those of ordinary skill in the art will know of methods and techniques for PEGylating a polymer, for example, by using EDC (I-ethyl-3-(3-dimethylaminopropyl) carbodiimide hydrochloride) and NHS (N-hydroxysuccinimide) to react a polymer to a PEG group terminating in an amine, by ring opening polymerization techniques (ROMP), or the like.

Other contemplated polymers that may form part of a disclosed nanoparticle may include poly(ortho ester) PEGylated poly(ortho ester), polylysine, PEGylated polylysine, poly(ethylene imine), PEGylated poly(ethylene imine), poly(L-lactide-co-L-lysine), poly(serine ester), poly(4-hydroxy-L-proline ester), poly[α-(4-aminobutyl)-L-glycolic acid], and derivatives thereof. In other embodiments, polymers can be degradable polyesters bearing cationic side chains. Examples of these polyesters include poly(L-lactide-co-L-lysine), poly(serine ester), poly(4-hydroxy-L-proline ester).

In other embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), methacrylic acid alkylamide copolymer, poly(methyl methacrylate), poly(methacrylic acid polyacrylamide, amino alkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

PLGA contemplated for use as described herein can be characterized by a lactic acid:glycolic acid ratio of e.g., approximately 85:15, approximately 75:25, approximately 60:40, approximately 50:50, approximately 40:60, approximately 25:75, or approximately 15:85. In some embodiments, the ratio of lactic acid to glycolic acid monomers in the polymer of the particle (e.g., a PLGA block copolymer or PLGA-PEG block copolymer), may be selected to optimize for various parameters such as water uptake, therapeutic agent release and/or polymer degradation kinetics can be optimized. In other embodiments, the end group of a PLA polymer chain may be a carboxylic acid group, an amine group, or a capped end group with e.g., a long chain alkyl group or cholesterol.

Particles disclosed herein may or may not contain PEG. In addition, certain embodiments can be directed towards copolymers containing poly(ester-ether)s, e.g., polymers having repeat units joined by ester bonds (e.g., R—C(O)—O—R′ bonds) and/or ether bonds (e.g., R—O—R′ bonds). Contemplated herein in certain embodiments is a biodegradable polymer, such as a hydrolyzable polymer containing carboxylic acid groups, that may be conjugated with poly(ethylene glycol) repeat units to form a poly(ester-ether).

In one embodiment, the molecular weight of the polymers can be optimized for effective treatment as disclosed herein. For example, the weight of a polymer may influence particle degradation rate (such as when the molecular weight of a biodegradable polymer can be adjusted), solubility, water uptake, and drug release kinetics. For example, the molecular weight of the polymer can be adjusted such that the particle biodegrades in the subject being treated within a reasonable period of time (ranging from a few hours to 1-2 weeks, 3-4 weeks, 5-6 weeks, 7-8 weeks, etc.) In an embodiment, a disclosed particle may comprise a copolymer of PEG and PLA, wherein the PEG portion may have a molecular weight of 1,000-20,000 g/mol, e.g., 5,000-20,000, e.g., 4,000-10,000 g/mol, and the PLA portion may have a molecular weight (for example, number average or weight average) of 5,000-100,000 g/mol, e.g., 10,000-80,000, e.g., 14,000-18,000 g/mol).

For example, disclosed biocompatible, therapeutic polymeric nanoparticle may include polylactic (acid)-polyethylene glycol co-polymer and/or polylactic (acid). Alternatively, a disclosed biocompatible, therapeutic polymeric nanoparticle may include polylactic-co-polyglycolic (acid)-polyethylene glycol co-polymer and/or polylactic-co-polyglycolic acid, or polycaprolactone and/or polycaprolactone-co-polyethylene glycol.

In an embodiment, a biocompatible, therapeutic polymeric nanoparticle contemplated herein may include a therapeutic agent, a PLA-PEG block copolymer or a PLGA-PEG block copolymer, and a glyceride such as a monoglyceride, a diglyceride, or a triglyceride. In an embodiment, the glyceride is not conjugated to PEG. The glyceride may be homogenously dispersed within the nanoparticle.

In an embodiment, a biocompatible, therapeutic polymeric nanoparticle contemplated herein may include a substantially hydrophobic boronate ester or boronate compound such as bortezomib, a PLA-PEG block copolymer or a PLGA-PEG block copolymer, and a glyceride. In an exemplary embodiment, the particles may include about 30% to about 40% by weight PLA-PEG block copolymer (e.g., PEG (5,000 Da)/PLA (16,000 Da)), about 10% to about 40% by weight glyceride, or about 20 to about 50 by weight glyceride, and about 20% to about 40% by weight boronate compound such as bortezomib. In another embodiment, the particles may include about 30% to about 40% by weight PLA-PEG block copolymer (e.g., PEG (5,000 Da)/PLA (50,000 Da)), about 10% to about 40% by weight glyceride, or about 20 to about 50 weight percent by weight glyceride, and about 20% to about 40% by weight boronate compound such as bortezomib.

In yet another embodiment, a biocompatible, therapeutic polymeric nanoparticle contemplated herein may include a taxane (for example, docetaxel). In an exemplary embodiment, the particles may include about 50% to about 70% by weight PLA-PEG block copolymer (e.g., PEG (5,000 Da)/PLA (16,000 Da)), about 5% to about 40% by weight glyceride, or about 10% to about 30% by weight glyceride, and about 20% to about 40% by weight a taxane (e.g. docetaxel). In another embodiment, the particles may include about 30% to about 40% by weight PLA-PEG block copolymer (e.g., PEG (5,000 Da)/PLA (50,000 Da)), about 30% to about 40% by weight glyceride, and about 20% to about 40% by weight a taxane such as docetaxel.

In general, any glyceride known in the art can be used in the invention. Contemplated glycerides include monoglycerides, diglycerides, and triglycerides.

Exemplary monoglycerides include, but are not limited to, lauroyl-rac-glycerol, glycerol monomyristate, glycerol monopalmitate, glycerol monostearate, glycerol monoarachidate, glycerol monobehenate, glycerol monopalmitoleate, glycerol monopalmitoleate, glycerol monooleate, glycerol monolinoleate, glycerol monolinolenate, glycerol monoarachidonate, glycerol monocaprylate, or combinations thereof.

Exemplary diglycerides include, but are not limited to, glycerol dilaurate, glycerol dimyristate, glycerol dipalmitate, glycerol distearate, glycerol diarachidate, glycerol dibehenate, glycerol dipalmitoleate, glycerl dioleate, glycerol dilinoleate, glycerol dilinolenate, glycerol diarachidonate, or combinations thereof.

Exemplary triglycerides include, but are not limited to, glycerol trilaurate, glycerol trimyristate, glycerol tripalmitate, glycerol tristearate, glycerol triarachidate, glycerol tribehenate, glycerol tripalmitoleate, glycerl trioleate, glycerol trilinoleate, glycerol trilinolenate, glycerol triarachidonate, or combinations thereof.

In some embodiments, disclosed therapeutic particles and/or compositions include targeting agents such as dyes, for example Evans blue dye. Such dyes may be bound to or associated with a therapeutic particle, or disclosed compositions may include such dyes. For example, Evans blue dye may be used, which may bind or associate with albumin, e.g. plasma albumin.

Disclosed therapeutic particles, may, some embodiments, include a targeting moiety, i.e., a moiety able to bind to or otherwise associate with a biological entity. The term “bind” or “binding,” as used herein, refers to the interaction between a corresponding pair of molecules or portions thereof that exhibit mutual affinity or binding capacity, typically due to specific or non-specific binding or interaction, including, but not limited to, biochemical, physiological, and/or chemical interactions. Therapeutic compositions disclosed herein may, for example, be locally administered to a designated region such as a blood vessel.

In certain embodiments, one or more polymers of a disclosed particle may be conjugated to a lipid. The polymer may be, for example, a lipid-terminated PEG. As described below, the lipid portion of the polymer can be used for self assembly with another polymer, facilitating the formation of a particle. For example, a hydrophilic polymer could be conjugated to a lipid that will self assemble with a hydrophobic polymer.

In some embodiments, lipids can be oils. In general, any oil known in the art can be conjugated to the polymers used in the invention. In some embodiments, an oil may comprise one or more fatty acid groups or salts thereof. In some embodiments, a fatty acid group may comprise digestible, long chain (e.g., C₈-C₅₀), substituted or unsubstituted hydrocarbons. In some embodiments, a fatty acid group may be a C₁₀-C₂₀ fatty acid or salt thereof. In some embodiments, a fatty acid group may be a C₁₅-C₂₀ fatty acid or salt thereof. In some embodiments, a fatty acid may be unsaturated. In some embodiments, a fatty acid group may be monounsaturated. In some embodiments, a fatty acid group may be polyunsaturated. In some embodiments, a double bond of an unsaturated fatty acid group may be in the cis conformation. In some embodiments, a double bond of an unsaturated fatty acid may be in the trans conformation.

In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palm itoleic, oleic, vaccenic, linoleic, alpha-linolenic, gamma-linoleic, arachidonic, gadoleic, arachidonic, eicosapentaenoic, docosahexaenoic, or erucic acid.

In one embodiment, the lipid can be of the Formula V:

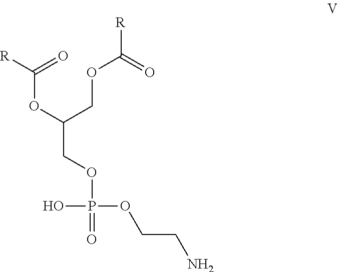

and salts thereof, wherein each R is, independently, C₁₋₃₀ alkyl. In one embodiment of Formula V, the lipid can be 1,2 distearoyl-sn-glycero-3-phosphoethanolamine (DSPE), and salts thereof, e.g., the sodium salt.

Also disclosed herein are compositions comprising a plurality of biocompatible, therapeutic polymeric nanoparticles as disclosed herein and a pharmaceutically acceptable excipient. Disclosed nanoparticles may have a substantially spherical (i.e., the particles generally appear to be spherical), or non-spherical configuration. For instance, the particles, upon swelling or shrinkage, may adopt a non-spherical configuration. In some cases, the particles may include polymeric blends. For instance, a polymer blend may include a first co-polymer that includes polyethylene glycol and a second polymer.

Disclosed nanoparticles may have a characteristic dimension of less than about 1 micrometer, where the characteristic dimension of a particle is the diameter of a perfect sphere having the same volume as the particle. For example, the particle can have a characteristic dimension of the particle can be less than about 300 nm, less than about 200 nm, less than about 150 nm, less than about 100 nm, less than about 50 nm, less than about 30 nm, less than about 10 nm, less than about 3 nm, or less than about 1 nm in some cases. In particular embodiments, disclosed nanoparticles may have a diameter of about 70 nm to about 200 nm, or about 70 nm to about 180 nm, about 80 nm to about 170nm, about 80 nm to about 130 nm.

In one set of embodiments, the particles can have an interior and a surface, where the surface has a composition different from the interior, i.e., there may be at least one compound present in the interior but not present on the surface (or vice versa), and/or at least one compound is present in the interior and on the surface at differing concentrations. For example, in one embodiment, a compound, such as a targeting moiety (i.e., a low-molecular weight ligand) of a polymeric conjugate of the present invention, may be present in both the interior and the surface of the particle, but at a higher concentration on the surface than in the interior of the particle, although in some cases, the concentration in the interior of the particle may be essentially nonzero, i.e., there is a detectable amount of the compound present in the interior of the particle.

In some cases, the interior of the particle is more hydrophobic than the surface of the particle. For instance, the interior of the particle may be relatively hydrophobic with respect to the surface of the particle, and a drug or other payload may be hydrophobic, and readily associates with the relatively hydrophobic center of the particle. The drug or other payload can thus be contained within the interior of the particle, which can shelter it from the external environment surrounding the particle (or vice versa). For instance, a drug or other payload contained within a particle administered to a subject will be protected from a subject's body, and the body may also be substantially isolated from the drug for at least a period of time.

For example, disclosed herein is a therapeutic polymeric nanoparticle comprising a first non-functionalized polymer; an optional second non-functionalized polymer; an optional functionalized polymer comprising a targeting moiety; and a therapeutic agent, In a particular embodiment, the first non-functionalized polymer is PLA, PLGA, or PEG, or copolymers thereof, e.g. a diblock co-polymer PLA-PEG. For example, exemplary nanoparticle may have a PEG corona with a density of about 0.065 g/cm³, or about 0.01 to about 0.10 g/cm³.

Disclosed nanoparticles may be stable, for example in a solution that may contain a saccharide, for at least about 24 hours, about 2 days, 3 days, about 4 days or at least about 5 days at room temperature, or at 25° C.

Nanoparticles may have controlled release properties, e.g., may be capable of delivering an amount of active agent to a patient, e.g., to specific site in a patient, over an extended period of time, e.g. over 1 day, 1 week, or more.

In one embodiment, the invention comprises a nanoparticle comprising 1) a polymeric matrix and 2) an amphiphilic compound or layer that surrounds or is dispersed within the polymeric matrix forming a continuous or discontinuous shell for the particle, An amphiphilic layer can reduce water penetration into the nanoparticle, thereby enhancing drug encapsulation efficiency and slowing drug release. Further, these amphiphilic layer protected nanoparticles can provide therapeutic advantages by releasing the encapsulated drug and polymer at appropriate times.

As used herein, the term “amphiphilic” refers to a property where a molecule has both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. Exemplary amphiphilic compound include, for example, one or a plurality of the following: naturally derived lipids, surfactants, or synthesized compounds with both hydrophilic and hydrophobic moieties.

Specific examples of amphiphilic compounds include, but are not limited to, phospholipids, such as 1,2 distearoyl-sn-glycero-3-phosphoethanolamine (DSPE), dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), and dilignoceroylphatidylcholine (DLPC), incorporated at a ratio of between 0.01-60 (weight lipid/w polymer), most preferably between 0.1-30 (weight lipid/w polymer). Phospholipids which may be used include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and β-acyl-y-alkyl phospholipids. Examples of phospholipids include, but are not limited to, phosphatidylcholines such as dioleoylphosphatidylcholine, dimyristoylphosphatidylcholine, dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcho-line (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophos-phoethanolamine. Synthetic phospholipids with asymmetric acyl chains (e.g., with one acyl chain of 6 carbons and another acyl chain of 12 carbons) may also be used.

In a particular embodiment, an amphiphilic component may include lecithin, and/or in particular, phosphatidylcholine.

### Preparation of Nanoparticles

Another aspect of the invention is directed to systems and methods of making disclosed nanoparticles. In some embodiments, by incorporating monoglycerides within the particles properties of particles may be controlled.

In an embodiment, provided herein is a method of preparing a plurality of biocompatible, therapeutic polymeric nanoparticles comprising: combining a therapeutic agent (e.g. docetaxel or bortezomib), a biodegradable polymer (e.g. PLA-PEG or PLGA-PEG), and a glyceride (e.g. a monoglyceride, a diglyceride, or a triglyceride) with an organic solution to form a first organic phase; combining the first organic phase with a first aqueous solution to form a second phase; emulsifying the second phase to form an emulsion phase; adding a drug solubilizer to the emulsion phase to form a solubilized phase; and recovering the biocompatible, therapeutic polymeric nanoparticles. In an embodiment, the glyceride is a monoglyceride (e.g. lauroyl-rac-glycerol). The glyceride may be homogenously dispersed within the nanoparticle.

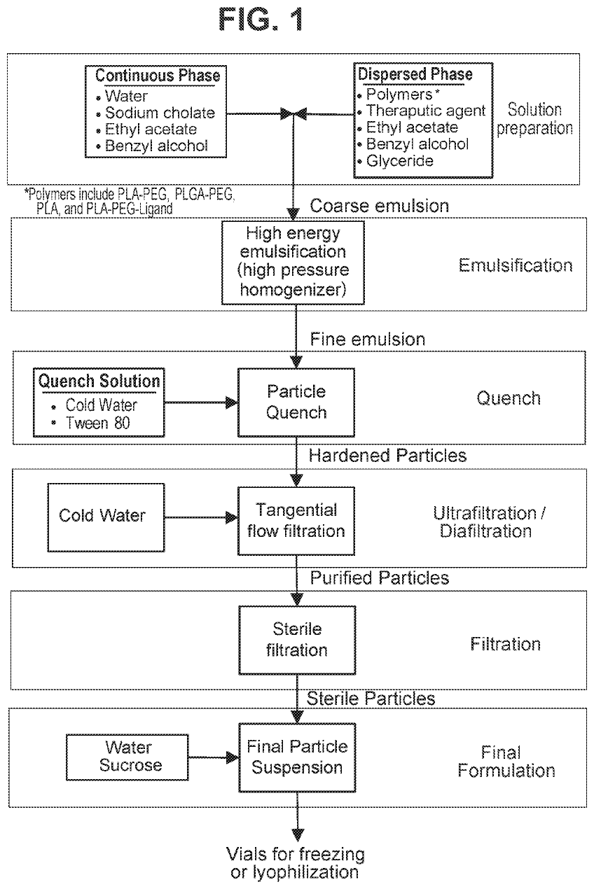

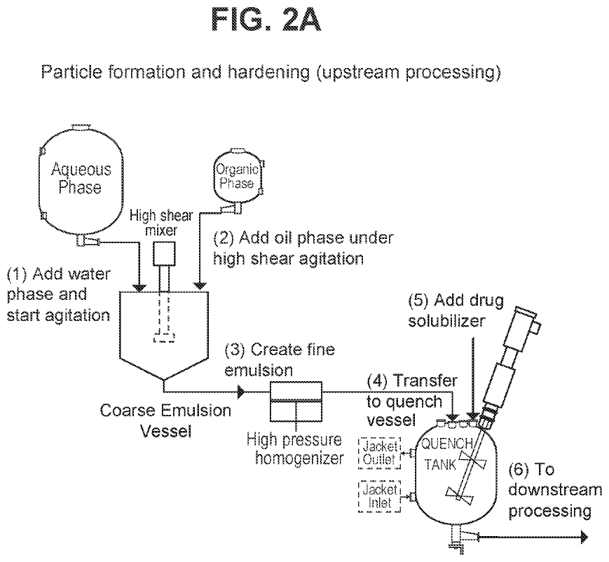

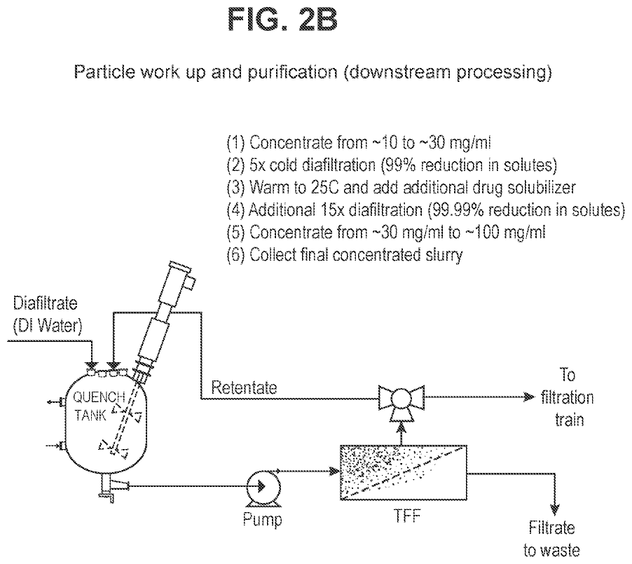

In an embodiment, a nanoemulsion process is provided, such as the process represented in FIGS. 1 and 2. For example, a therapeutic agent, a monoglyceride, a first polymer (for example, PLA-PEG or PLGA-PEG) and/or a second polymer (e.g. (PL(G)A or PLA), is mixed with an organic solution to form a first organic phase. Such first phase may include about 5 to about 50% weight solids, e.g. about 5 to about 40% solids, or about 10 to about 30% solids, e.g. about 10%, 15%, 20% solids. The first organic phase may be combined with a first aqueous solution to form a second phase. The organic solution can include, for example, acetonitrile, tetrahydrofuran, ethyl acetate, isopropyl alcohol, isopropyl acetate, dimethylformamide, methylene chloride, dichloromethane, chloroform, acetone, benzyl alcohol, Tween 80, Span 80,or the like, and combinations thereof. In an embodiment, the organic phase may include benzyl alcohol, ethyl acetate, and combinations thereof. The second phase can be between about 1 and 50 weight % , e.g., 5-40 weight %, solids. The aqueous solution can be water, optionally in combination with one or more of sodium cholate, ethyl acetate, and benzyl alcohol.

For example, the oil or organic phase may use solvent that is only partially miscible with the nonsolvent (water). Therefore, when mixed at a low enough ratio and/or when using water pre-saturated with the organic solvents, the oil phase remains liquid. The oil phase may be emulsified into an aqueous solution and, as liquid droplets, sheared into nanoparticles using, for example, high energy dispersion systems, such as homogenizers or sonicators. The aqueous portion of the emulsion, otherwise known as the “water phase”, may be surfactant solution consisting of sodium cholate and pre-saturated with ethyl acetate and benzyl alcohol.

Emulsifying the second phase to form an emulsion phase may be performed in one or two emulsification steps. For example, a primary emulsion may be prepared, and then emulsified to form a fine emulsion. The primary emulsion can be formed, for example, using simple mixing, a high pressure homogenizer, probe sonicator, stir bar, or a rotor stator homogenizer. The primary emulsion may be formed into a fine emulsion through the use of e.g. probe sonicator or a high pressure homogenizer, e.g. by using 1, 2, 3 or more passes through a homogenizer. For example, when a high pressure homogenizer is used, the pressure used may be about 4000 to about 8000 psi, or about 4000 to about 5000 psi, e.g. 4000 or 5000 psi.

Either solvent evaporation or dilution may be needed to complete the extraction of the solvent and solidify the particles. For better control over the kinetics of extraction and a more scalable process, a solvent dilution via aqueous quench may be used. For example, the emulsion can be diluted into cold water to a concentration sufficient to dissolve all of the organic solvent to form a quenched phase. Quenching may be performed at least partially at a temperature of about 5° C. or less. For example, water used in the quenching may be at a temperature that is less that room temperature (e.g. about 0 to about 10° C., or about 0 to about 5° C.).

In some embodiments, not all of the therapeutic agent is encapsulated in the particles at this stage, and a drug solubilizer is added to the quenched phase to form a solubilized phase. The drug solubilizer may be for example, Tween 80, Tween 20, polyvinyl pyrrolidone, cyclodextran, sodium dodecyl sulfate, or sodium cholate. For example, Tween-80 may added to the quenched nanoparticle suspension to solubilize the free drug and prevent the formation of drug crystals. In some embodiments, a ratio of drug solubilizer to therapeutic agent is about 100:1 to about 10:1.

The solubilized phase may be filtered to recover the nanoparticles. For example, ultrafiltration membranes may be used to concentrate the nanoparticle suspension and substantially eliminate organic solvent, free drug, and other processing aids (surfactants). Exemplary filtration may be performed using a tangential flow filtration system. For example, by using a membrane with a pore size suitable to retain nanoparticles while allowing solutes, micelles, and organic solvent to pass, nanoparticles can be selectively separated. Exemplary membranes with molecular weight cut-offs of about 300-500 kDa (˜5-25 nm) may be used.

Diafiltration may be performed using a constant volume approach, meaning the diafiltrate (cold deionized water, e.g. about 0° C. to about 5° C., or 0 to about 10° C.) may added to the feed suspension at the same rate as the filtrate is removed from the suspension. In some embodiments, filtering may include a first filtering using a first temperature of about 0° C. to about 5° C., or 0° C. to about 10° C., and a second temperature of about 20° C. to about 30° C., or 15° C. to about 35° C. For example, filtering may include processing about 1 to about 6 diavolumes at about 0° C. to about 5° C., and processing at least one diavolume (e.g. about 1 to about 3 or about 1-2 diavolumes) at about 20° C. to about 30° C.

After purifying and concentrating the nanoparticle suspension, the particles may be passed through one, two or more sterilizing and/or depth filters, for example, using ˜0.2 μm depth pre-filter.

In exemplary embodiment of preparing nanoparticles, an organic phase is formed composed of a mixture of a therapeutic agent, e.g., docetaxel or bortezomib, a glyceride (e.g. a monoglyceride, a diglyceride, or a triglyceride), and polymer (PLA-PEG or PLGA-PEG). The organic phase may be mixed with an aqueous phase at approximately a 1:5 ratio (oil phase:aqueous phase) where the aqueous phase is composed of a surfactant and optionally dissolved solvent. A primary emulsion may then formed by the combination of the two phases under simple mixing or through the use of a rotor stator homogenizer. The primary emulsion is then formed into a fine emulsion through the use of e.g. high pressure homogenizer. Such fine emulsion may then quenched by, e.g. addition to deionized water under mixing. An exemplary quench:emulsion ratio may be about approximately 8:1. A solution of Tween (e.g., Tween 80) can then be added to the quench to achieve e.g. approximately 2% Tween overall, which may serve to dissolve free, unencapsulated drug. Formed nanoparticles may then be isolated through either centrifugation or ultrafiltration/diafiltration.

### Therapeutic Agents

According to the present invention, any agents including, for example, therapeutic agents (e.g. anti-cancer agents), diagnostic agents (e.g. contrast agents; radionuclides; and fluorescent, luminescent, and magnetic moieties), prophylactic agents (e.g. vaccines), and/or nutraceutical agents (e.g. vitamins, minerals, etc.) may be delivered by the disclosed nanoparticles. Exemplary agents to be delivered in accordance with the present invention include, but are not limited to, small molecules (e.g. cytotoxic agents), nucleic acids (e.g., siRNA, RNAi, and mircoRNA agents), proteins (e.g. antibodies), peptides, lipids, carbohydrates, hormones, metals, radioactive elements and compounds, drugs, vaccines, immunological agents, etc., and/or combinations thereof. In some embodiments, the agent to be delivered is an agent useful in the treatment of cancer (e.g., prostate cancer or hematologic malignancy). The active agent or drug may be a therapeutic agent such as mTor inhibitors (e.g., sirolimus, temsirolimus, or everolimus), vinca alkaloids (e.g. vinorelbine or vincristine), a diterpene derivative, a taxane (e.g. paclitaxel or its derivatives such as DHA-paclitaxel or PG-paxlitaxelor, or docetaxel), a boronate ester or peptide boronic acid compound (e.g. bortezomib), a cardiovascular agent (e.g. a diuretic, a vasodilator, angiotensin converting enzyme, a beta blocker, an aldosterone antagonist, or a blood thinner), a corticosteroid, an antimetabolite or antifolate agent (e.g. methotrexate), a chemotherapeutic agent (e.g. epothilone B), an alkylating agent (e.g. bendamustine), or the active agent or drug may be an siRNA.

In one set of embodiments, the payload is a drug or a combination of more than one drug. Such particles may be useful, for example, in embodiments where a targeting moiety may be used to direct a particle containing a drug to a particular localized location within a subject, e.g., to allow localized delivery of the drug to occur. Exemplary therapeutic agents include chemotherapeutic agents such as doxorubicin (adriamycin), gemcitabine (gemzar), daunorubicin, procarbazine, mitomycin, cytarabine, etoposide, methotrexate, venorelbine, 5-fluorouracil (5-FU), vinca alkaloids such as vinblastine or vincristine; bleomycin, paclitaxel (taxol), docetaxel (taxotere), aldesleukin, asparaginase, bortezomib, busulfan, carboplatin, cladribine, camptothecin, CPT-11, 10-hydroxy-7-ethylcamptothecin (SN38), dacarbazine, S-I capecitabine, ftorafur, 5′deoxyflurouridine, UFT, eniluracil, deoxycytidine, 5-azacytosine, 5-azadeoxycytosine, allopurinol, 2-chloroadenosine, trimetrexate, aminopterin, methylene-10-deazaam inopterin (MDAM), oxaplatin, picoplatin, tetraplatin, satraplatin, platinum-DACH, ormaplatin, CI-973, JM-216, and analogs thereof, epirubicin, etoposide phosphate, 9-aminocamptothecin, 10,11-methylenedioxycamptothecin, karenitecin, 9-nitrocamptothecin, TAS 103, vindesine, L-phenylalanine mustard, ifosphamidemefosphamide, perfosfamide, trophosphamide carmustine, semustine, epothilones A-E, tomudex, 6-mercaptopurine, 6-thioguanine, amsacrine, etoposide phosphate, karenitecin, acyclovir, valacyclovir, ganciclovir, amantadine, rimantadine, lamivudine, zidovudine, bevacizumab, trastuzumab, rituximab, 5-Fluorouracil, methotrexate, budesonide, sirolimus vincristine, and combinations thereof, or the therapeutic agent may be an siRNA

Non-limiting examples of potentially suitable drugs include anti-cancer agents, including, for example, docetaxel, mitoxantrone, and mitoxantrone hydrochloride. In another embodiment, the payload may be an anti-cancer drug such as 20-epi-1, 25 dihydroxyvitam in D3, 4-ipomeanol, 5-ethynyluracil, 9-dihydrotaxol, abiraterone, acivicin, aclarubicin, acodazole hydrochloride, acronine, acylfiilvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizdng morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisazuidinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caraceraide, carbetimer, carboplatin, carboxamide-amino-triazole, carboxyamidotriazole, carest M3, carmustine, earn 700, cartilage derived inhibitor, carubicin hydrochloride, carzelesin, casein kinase inhibitors, castanosperrnine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethyhiorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocannycin SA, ebselen, ecomustine, edatrexate, edelfosine, edrecolomab, eflomithine, eflomithine hydrochloride, elemene, elsarnitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, flurocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ihnofosine, ilomastat, imidazoacridones, imiquimod, immunostimulant peptides, insulin-like growth factor-1 receptor inhibitor, interferon agonists, interferon alpha-2A, interferon alpha-2B, interferon alpha-NI, interferon alpha-N3, interferon beta-IA, interferon gamma-IB, interferons, interleukins, iobenguane, iododoxorubicin, iproplatm, irinotecan, irinotecan hydrochloride, iroplact, irsogladine, isobengazole, isohomohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide, lobaplatin, lombricine, lometrexol, lometrexol sodium, lomustine, lonidamine, losoxantrone, losoxantrone hydrochloride, lovastatin, loxoribine, lurtotecan, lutetium texaphyrin lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C uihibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, O6-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazorurin, pyrazoloacridine, pyridoxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RH retinarnide, RNAi, rogletimide, rohitukine, romurtide, roquinimex, rubiginone BI, ruboxyl, safingol, safingol hydrochloride, saintopin, sarcnu, sarcophytol A, sargramostim, SDI1 mimetics, semustine, senescence derived inhibitor 1, sense oligonucleotides, signal transduction inhibitors, signal transduction modulators, simtrazene, single chain antigen binding protein, sizofiran, sobuzoxane, sodium borocaptate, sodium phenylacetate, solverol, somatomedin binding protein, sonermin, sparfosafe sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine or vinorelbine tartrate, vinrosidine sulfate, vinxaltine, vinzolidine sulfate, vitaxin, vorozole, zanoterone, zeniplatin, zilascorb, zinostatin, zinostatin stimalamer, or zorubicin hydrochloride.

### Compositions and Methods of Treatment

Nanoparticles disclosed herein may be combined with pharmaceutical acceptable carriers to form a pharmaceutical composition. As would be appreciated by one of skill in this art, the carriers may be chosen based on the route of administration as described below, the location of the target issue, the drug being delivered, the time course of delivery of the drug, etc.

The pharmaceutical compositions and particles disclosed herein can be administered to a patient by any means known in the art including oral and parenteral routes. The term “patient,” as used herein, refers to humans as well as non-humans, including, for example, mammals, birds, reptiles, amphibians, and fish. For instance, the non-humans may be mammals (e.g., a rodent, a mouse, a rat, a rabbit, a monkey, a dog, a cat, a primate, or a pig). In certain embodiments parenteral routes are desirable since they avoid contact with the digestive enzymes that are found in the alimentary canal. According to such embodiments, inventive compositions may be administered by injection (e.g., intravenous, subcutaneous or intramuscular, intraperitoneal injection), rectally, vaginally, topically (as by powders, creams, ointments, or drops), or by inhalation (as by sprays).

In a particular embodiment, disclosed nanoparticles may be administered to a subject in need thereof systemically, e.g., by IV infusion or injection.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In one embodiment, the inventive conjugate is suspended in a carrier fluid comprising 1% (w/v) sodium carboxymethyl cellulose and 0.1% (v/v) TWEEN™ 80. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the encapsulated or unencapsulated conjugate is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Disclosed nanoparticles may be formulated in dosage unit form for ease of administration and uniformity of dosage. The expression “dosage unit form” as used herein refers to a physically discrete unit of nanoparticle appropriate for the patient to be treated. For any nanoparticle, the therapeutically effective dose can be estimated initially either in cell culture assays or in animal models, usually mice, rabbits, dogs, or pigs. An animal model may also be used to achieve a desirable concentration range and route of administration. Such information can then be used to determine useful doses and routes for administration in humans. Therapeutic efficacy and toxicity of nanoparticles can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices may be useful in some embodiments. The data obtained from cell culture assays and animal studies can be used in formulating a range of dosage for human use.

In an exemplary embodiment, a pharmaceutical composition is disclosed that includes a plurality of nanoparticles each comprising a therapeutic agent and a pharmaceutically acceptable excipient.

In some embodiments, a composition suitable for freezing is contemplated, including nanoparticles disclosed herein and a solution suitable for freezing, e.g., a sugar (e.g. sucrose) solution is added to a nanoparticle suspension. The sucrose may, e.g., act as a cryoprotectant to prevent the particles from aggregating upon freezing. For example, provided herein is a nanoparticle formulation comprising a plurality of disclosed nanoparticles, sucrose and water; wherein, for example, the nanoparticles/sucrose/water are present at about 5-10%/10-15%/80-90% (w/w/w).

In some embodiments, therapeutic particles disclosed herein may be used to treat, alleviate, ameliorate, relieve, delay onset of, inhibit progression of, reduce severity of, and/or reduce incidence of one or more symptoms or features of a disease, disorder, and/or condition. For example, disclosed therapeutic particles, that include taxane, e.g., docetaxel, may be used to treat cancers such as breast or prostate cancer in a patient in need thereof. Other types of tumors and cancer cells to be treated with therapeutic particles of the present invention include all types of solid tumors, such as those which are associated with the following types of cancers: lung, squamous cell carcinoma of the head and neck (SCCHN), pancreatic, colon, rectal, esophageal, prostate, breast, ovarian carcinoma, renal carcinoma, lymphoma and melanoma. The tumor can be associated with cancers of (i.e., located in) the oral cavity and pharynx, the digestive system, the respiratory system, bones and joints (e.g., bony metastases), soft tissue, the skin (e.g., melanoma), breast, the genital system, the urinary system, the eye and orbit, the brain and nervous system (e.g., glioma), or the endocrine system (e.g., thyroid) and is not necessarily the primary tumor. Tissues associated with the oral cavity include, but are not limited to, the tongue and tissues of the mouth. Cancer can arise in tissues of the digestive system including, for example, the esophagus, stomach, small intestine, colon, rectum, anus, liver, gall bladder, and pancreas. Cancers of the respiratory system can affect the larynx, lung, and bronchus and include, for example, non-small cell lung carcinoma. Tumors can arise in the uterine cervix, uterine corpus, ovary vulva, vagina, prostate, testis, and penis, which make up the male and female genital systems, and the urinary bladder, kidney, renal pelvis, and ureter, which comprise the urinary system.

Disclosed methods for the treatment of cancer (e.g. breast or prostate cancer) may comprise administering a therapeutically effective amount of the disclosed therapeutic particles to a subject in need thereof, in such amounts and for such time as is necessary to achieve the desired result. In certain embodiments of the present invention a “therapeutically effective amount” is that amount effective for treating, alleviating, ameliorating, relieving, delaying onset of, inhibiting progression of, reducing severity of, and/or reducing incidence of one or more symptoms or features of e.g. a cancer being treated.

Also provided herein are therapeutic protocols that include administering a therapeutically effective amount of an disclosed therapeutic particle to a healthy individual (i.e., a subject who does not display any symptoms of cancer and/or who has not been diagnosed with cancer). For example, healthy individuals may be “immunized” with an inventive targeted particle prior to development of cancer and/or onset of symptoms of cancer; at risk individuals (e.g., patients who have a family history of cancer; patients carrying one or more genetic mutations associated with development of cancer; patients having a genetic polymorphism associated with development of cancer; patients infected by a virus associated with development of cancer; patients with habits and/or lifestyles associated with development of cancer; etc.) can be treated substantially contemporaneously with (e.g., within 48 hours, within 24 hours, or within 12 hours of) the onset of symptoms of cancer. Of course individuals known to have cancer may receive inventive treatment at any time.

In other embodiments, disclosed nanoparticles may be used to inhibit the growth of cancer cells, e.g., breast cancer cells. As used herein, the term “inhibits growth of cancer cells” or “inhibiting growth of cancer cells” refers to any slowing of the rate of cancer cell proliferation and/or migration, arrest of cancer cell proliferation and/or migration, or killing of cancer cells, such that the rate of cancer cell growth is reduced in comparison with the observed or predicted rate of growth of an untreated control cancer cell. The term “inhibits growth” can also refer to a reduction in size or disappearance of a cancer cell or tumor, as well as to a reduction in its metastatic potential. Preferably, such an inhibition at the cellular level may reduce the size, deter the growth, reduce the aggressiveness, or prevent or inhibit metastasis of a cancer in a patient. Those skilled in the art can readily determine, by any of a variety of suitable indicia, whether cancer cell growth is inhibited.

Inhibition of cancer cell growth may be evidenced, for example, by arrest of cancer cells in a particular phase of the cell cycle, e.g., arrest at the G2/M phase of the cell cycle. Inhibition of cancer cell growth can also be evidenced by direct or indirect measurement of cancer cell or tumor size. In human cancer patients, such measurements generally are made using well known imaging methods such as magnetic resonance imaging, computerized axial tomography and X-rays. Cancer cell growth can also be determined indirectly, such as by determining the levels of circulating carcinoembryonic antigen, prostate specific antigen or other cancer-specific antigens that are correlated with cancer cell growth. Inhibition of cancer growth is also generally correlated with prolonged survival and/or increased health and well-being of the subject.

### EXAMPLES

The invention now being generally described, it will be more readily understood by reference to the following examples which are included merely for purposes of illustration of certain aspects and embodiments of the present invention, and are not intended to limit the invention in any way.

### Example 1

### Preparation of PLA-PEG

The synthesis is accomplished by ring opening polymerization of d,l-lactide with α-hydroxy-ω-methoxypoly(ethylene glycol) as the macro-initiator, and performed at an elevated temperature using Tin (II) 2-Ethyl hexanoate as a catalyst, as shown below (PEG Mn≈5,000 Da; PLA Mn≈16,000 Da; PEG-PLA Mₙ≈21,000 Da).

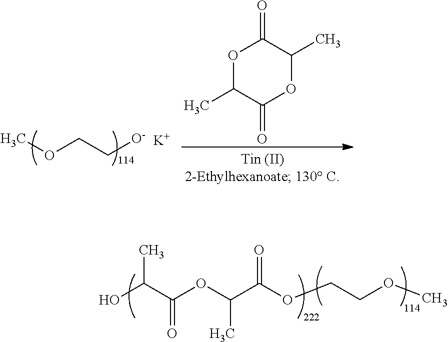

The polymer is purified by dissolving the polymer in dichloromethane, and precipitating it in a mixture of hexane and diethyl ether. The polymer recovered from this step is dried in an oven.

### Example 2

### Bortezomib Nanoparticles

Bortezomib nanoparticles were prepared by blending PLA-PEG copolymer with monoglycerol lipids using the following formulation: 30% theoretical (w/w) drug; 70% (w/w) polymer-PEG (16/5 PLA-PEG or 50/5 PLA-PEG) and lipid (monoglyceride). % Total solids=20%; solvents: 21% benzyl alcohol and 79% ethyl acetate (w/w). For a 1 gram batch size, 300 mg of drug was mixed with 700 mg of a blend of Polymer-PEG (16-5 or 50-5 PLA-PEG) and lipid.

Bortezomib nanoparticles comprising monoglycerol lipids were produced as follows. In order to prepare a drug/polymer solution, appropriate amounts of bortezomib, polymer, and lipids were added to a 25 mL glass vial along with 3.16 g of ethyl acetate and 0.84 g of benzyl alcohol. The mixture was vortexed until the drug, polymer, and lipids were completely dissolved.

An aqueous solution for either a 16-5 PLA-PEG formulation or a 50-5 PLA-PEG formulation was prepared. The 16-5 PLA-PEG formulation contained 0.05% sodium cholate, 2% benzyl alcohol, and 4% ethyl acetate in water. Specifically, 0.5 g of sodium cholate and 939.5 g of DI water were added to a 1 L bottle and mixed using a stir plate until they were dissolved. Subsequently, 20 g of benzyl alcohol and 40 g of ethyl acetate were added to the sodium cholate/water mixture and mixed using a stir plate until all were dissolved. The 50-5 formulation contained 0.25% sodium cholate, 2% benzyl alcohol, and 4% ethyl acetate in water. Specifically, 2.5 g of sodium cholate and 937.5 g of DI water were added to a 1 L bottle and mixed using a stir plate until they were dissolved. Subsequently, 20 g of benzyl alcohol and 40 g of ethyl acetate were added to the sodium cholate/water mixture and mixed using a stir plate until all were dissolved.

An emulsion was formed by combining the organic phase into the aqueous solution at a ratio of 5:1 (aqueous phase:oil phase). The organic phase was poured into the aqueous solution and homogenized using hand homogenizer for 10 seconds at room temperature to form a coarse emulsion. The solution was subsequently fed through a high pressure homogenizer (110S). For the 16-5 PLA-PEG formulation, the pressure was set to 45 psi on gauge for two discreet passes to form the nanoemulsion. For the 50-5 PLA-PEG formulation, the pressure was set to 45 psi on gauge for two to four discreet passes to form the nanoemulsion.

The emulsion was quenched into cold DI water at <5° C. while stirring on a stir plate. The ratio of Quench to Emulsion was 8:1. 35% (w/w) Tween 80 in water was then added to the quenched emulsion at a ratio of 25:1 (Tween 80:drug).

The nanoparticles were concentrated through tangential flow filtration (TFF) followed by diafiltration to remove solvents, unencapsulated drug and solubilizer. A quenched emulsion was initially concentrated through TFF using a 300 KDa Pall cassette (2 membrane) to an approximately 100 mL volume. This was followed by diafiltration using approximately 20 diavolumes (2 L) of cold DI water. The volume was minimized by adding 100 mL of cold water to the vessel and pumping through the membrane for rinsing. Approximately 100-180 mL of material were collected in a glass vial. The nanoparticles were further concentrated using a smaller TFF to a final volume of approximately 10-20 mL.

In order to determine the solids concentration of unfiltered final slurry, a volume of final slurry was added to a tared 20 mL scintillation vial and dried under vacuum on lyo/oven. Subsequently the weight of nanoparticles was determined in the volume of the dried down slurry. Concentrated sucrose (0.666 g/g) was added to the final slurry sample to attain a final concentration of 10% sucrose.

In order to determine the solids concentration of 0.45 μm filtered final slurry, a portion of the final slurry sample was filtered before the addition of sucrose using a 0.45 μm syringe filter. A volume of the filtered sample was then added to a tared 20 mL scintillation vial and dried under vacuum on lyo/oven. The remaining sample of unfiltered final slurry were frozen with sucrose.

The following batches of bortezomib nanoparticles were produced, as shown in Table A.

TABLE A

| | 35% Lipid + 35% 16/5 | | | | | |
|---|---|---|---|---|---|---|
| | PLA/PEG + 30% BTZ | | 35% Lipid + 35% 50/5 PLA/PEG + 30% BTZ | | | |
| Lot # | 82-130-2 | 82-170-6 | 82-170-1 | 82-170-2 | 82-170-3 | 82-180-2A |

| | | |
|---|---|---|
| BTZ | 30% (300 mg) | 30% (300 mg) |
| Polymer | 35% (35 0mg) 16/5 | 35% (350 mg) 50/5 PLA/PEG |
| PLA/PEG | PLA/PEG | |
| Lauroyl-rac- | 35% (350 mg) | 35% (35 mg) |
| Glycerol | | |

Table B provides the particle size and drug load of the bortezomib nanoparticles described above.

TABLE B

| | 35% Lipid + 35% 16/5 | | | | | |
|---|---|---|---|---|---|---|
| | PLA/PEG + 30% BTZ | | 35% Lipid + 35% 50/5 PLA/PEG + 30% BTZ | | | |
| Lot# | 82-130-2 | 82-170-6 | 82-170-1 | 82-170-2 | 82-170-3 | 82-180-2A |
| Load (%) | 18.06 | 12.12 | 2.46 | 5.34 | 3.83 | 2.46 |
| Size (nm) | 117.70 | 146.90 | 139.90 | 169.90 | 153.30 | 126.40 |

Incorporation of 16-5 PLA-PEG and the monoglycerol lipid, lauroyl-rac-glycerol, into bortezomib nanoparticles appeared to result in higher drug encapsulation efficiency and higher drug loading. As shown in Table B, bortezomib nanoparticles comprising 16-5 PLA-PEG and lauroyl-rac-glycerol resulted in a drug load of more than 12%.

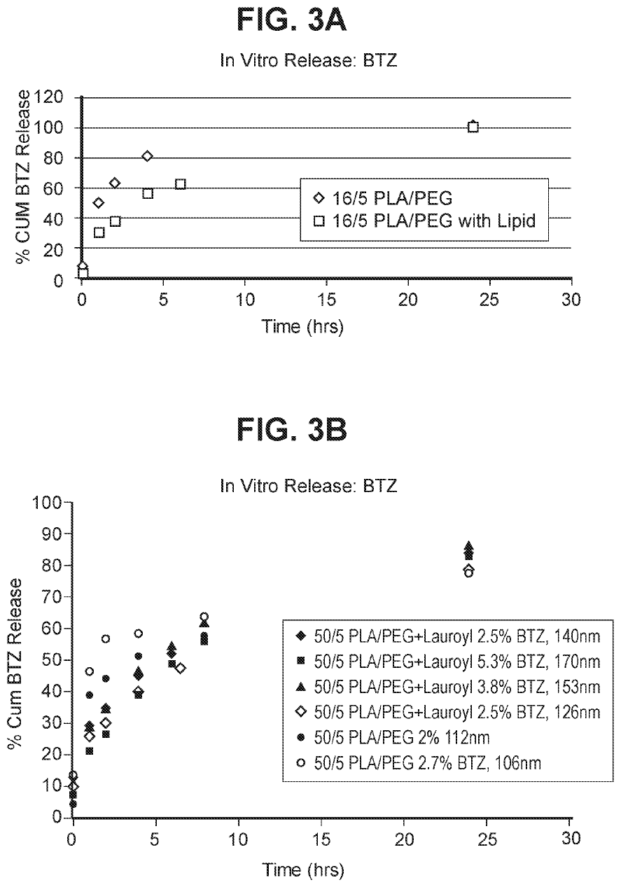

In vitro release test is performed on the above described bortezomib nanoparticles. As depicted in FIGS. 3A and 3B, incorporation of either the 16-5 PLA-PEG or 50-5 PLA-PEG in combination with lauroyl-rac-glycerol slowed down the release of bortezomib from the nanoparticles compared with nanoparticles without lipids.

### Example 3

### Docetaxel Nanoparticles

Docetaxel nanoparticles were prepared by blending PLA-PEG copolymer with monoglycerol lipids using the following formulation: 30% theoretical (w/w) drug; 70% (w/w) polymer-PEG (16/5 PLA-PEG) and lipid (monoglyceride). % Total solids=20%; solvents: 21% benzyl alcohol and 79% ethyl acetate (w/w). For a 2 gram batch size, 600 mg of drug was mixed with 1400 mg of a blend of Polymer-PEG (16-5 PLA-PEG) and lipid.

Docetaxel nanoparticles comprising monoglycerol lipids were produced as follows. In order to prepare a drug/polymer solution, appropriate amounts of docetaxel, polymer, and lipids were added to a 25 mL glass vial along with 6.32 g of ethyl acetate and 1.68 g of benzyl alcohol. The mixture was vortexed until the drug, polymer, and lipids were completely dissolved. The docetaxel nanoparticles comprised about 10 to about 35 weight percent of the monoglycerol lipid, lauroyl-rac-glycerol.

An aqueous solution was prepared. The 16-5 PLA-PEG formulation contained 0.05% sodium cholate, 2% benzyl alcohol, and 4% ethyl acetate in water. Specifically, 0.5 g of sodium cholate and 939.5 g of DI water were added to a 1 L bottle and mixed using a stir plate until they were dissolved. Subsequently, 20 g of benzyl alcohol and 40 g of ethyl acetate were added to the sodium cholate/water mixture and mixed using a stir plate until all were dissolved.

An emulsion was formed by combining the organic phase into the aqueous solution at a ratio of 5:1 (aqueous phase:oil phase). The organic phase was poured into the aqueous solution and homogenized using hand homogenizer for 10 seconds at room temperature to form a coarse emulsion. The solution was subsequently fed through a high pressure homogenizer (110S). The pressure was set to 45 psi on gauge for two discreet passes to form the nanoemulsion.

The emulsion was quenched into cold DI water at <5° C. while stirring on a stir plate. The ratio of Quench to Emulsion was 8:1. 35% (w/w) Tween 80 in water was then added to the quenched emulsion at a ratio of 25:1 (Tween 80:drug).

The nanoparticles were concentrated through tangential flow filtration (TFF) followed by diafiltration to remove solvents, unencapsulated drug and solubilizer. A quenched emulsion was initially concentrated through TFF using a 300 KDa Pall cassette (2 membrane) to an approximately 100 mL volume. This was followed by diafiltration using approximately 20 diavolumes (2 L) of cold DI water. The volume was minimized by adding 100 mL of cold water to the vessel and pumping through the membrane for rinsing. Approximately 100-180 mL of material were collected in a glass vial. The nanoparticles were further concentrated using a smaller TFF to a final volume of approximately 10-20 mL.

In order to determine the solids concentration of unfiltered final slurry, a volume of final slurry was added to a tared 20 mL scintillation vial and dried under vacuum on lyo/oven. Subsequently the weight of nanoparticles was determined in the volume of the dried down slurry. Concentrated sucrose (0.666 g/g) was added to the final slurry sample to attain a final concentration of 10% sucrose.

In order to determine the solids concentration of 0.45 μm filtered final slurry, a portion of the final slurry sample was filtered before the addition of sucrose using a 0.45 μm syringe filter. A volume of the filtered sample was then added to a tared 20 mL scintillation vial and dried under vacuum on lyo/oven. The remaining sample of unfiltered final slurry were frozen with sucrose.

The following batches of docetaxel nanoparticles were produced, as shown in Table C.

TABLE C

| | 10% Lipid + 60% | | | | | |
|---|---|---|---|---|---|---|
| | PLA/PEG + 30% DTXL | | | 35% Lipid + 35% PLA/PEG + 30% DTXL | | |
| Lot # | 82-106-1B | 82-106-1C | 82-106-1D | 82-106-2B | 82-106-20 | 82-106-2D |

| | | |
|---|---|---|
| DTXL | 30% (600 mg) | 30% (600 mg) |
| 16/5 | 60% (1200 mg) | 35% (700 mg) |
| PLA/PEG | | |
| Lauroyl-rac- | 10% (200 mg) | 35% (700 mg) |
| Glycerol | | |

Table D provides the particle size and drug load of the docetaxel nanoparticles described above.

TABLE D

| | 82-106-1B | 82-106-1C | 82-106-1D | 82-106-2B | 82-106-2C | 82-106-2D |
|---|---|---|---|---|---|---|
| Drug load (%) | 10.26 | 11.74 | 10.84 | 9.87 | 10.59 | 10.20 |
| Size (nm) | 108.10 | 106.40 | 110.50 | 121.70 | 113.10 | 117.50 |

As shown in Table D, docetaxel nanoparticles comprising 16-5 PLA-PEG and lauroyl-rac-glycerol resulted in a drug load of from about 9.9% to about 11.7%.

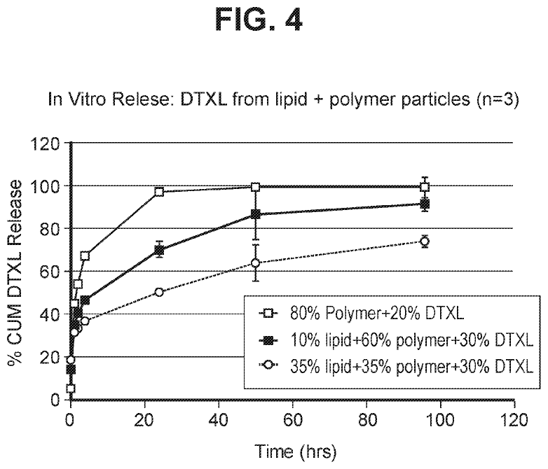

In vitro release test is performed on the above described docetaxel nanoparticles. As depicted in FIG. 4, incorporation of 16-5 PLA-PEG in combination with lauroyl-rac-glycerol slowed down the release of docetaxel from the nanoparticles compared with nanoparticles without lipids.

### Equivalents

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

The entire contents of all patents, published patent applications, websites, and other references cited herein are hereby expressly incorporated herein in their entireties by reference.

1. A biocompatible, therapeutic polymeric nanoparticle comprising:
about 10 to about 70 weight percent a biodegradable polymer;
about 5 to about 50 weight percent glyceride; and
about 0.1 to about 40 weight percent of a therapeutic agent selected from a taxane and bortezomib.

2. The biocompatible, therapeutic polymeric nanoparticle of claim 1, wherein the biodegradable polymer comprises a diblock poly(lactic) acid-poly(ethylene)glycol copolymer or a diblock poly(lactic)-co-poly (glycolic) acid-poly(ethylene)glycol copolymer.

3. The biocompatible, therapeutic nanoparticle of claim 2, wherein the polyethylene glycol portion has a molecular weight of about 4 kDa to about 6 kDa.

4. The biocompatible, therapeutic nanoparticle of claim 2 or 3, wherein the polylactic acid portion has a molecular weight of about 12 kDa to about 80 kDa.

5. The biocompatible, therapeutic polymeric nanoparticle of claim 1, wherein the glyceride is selected from a group consisting of monoglyceride, diglyceride, and triglyceride.

6. The biocompatible, therapeutic polymeric nanoparticle of claim 5, wherein the glyceride is a monoglyceride.

7. The biocompatible, therapeutic polymeric nanoparticle of claim 6, wherein the monoglyceride comprises lauroyl-rac-glycerol, glycerol monomyristate, glycerol monopalmitate, glycerol monostearate, glycerol monoarachidate, glycerol monobehenate, glycerol monopalmitoleate, glycerol monopalmitoleate, glycerol monooleate, glycerol monolinoleate, glycerol monolinolenate, glycerol monoarachidonate, glycerol monocaprylate, or combinations thereof.

8. The biocompatible, therapeutic polymeric nanoparticle of claim 7, wherein the monoglyceride is lauroyl-rac-glycerol.

9. The biocompatible, therapeutic polymeric nanoparticle of any one of claims 1-8, wherein the glyceride is homogenously dispersed within the nanoparticle.

10. The biocompatible, therapeutic polymeric nanoparticle of claim 1, wherein the therapeutic agent is a taxane.

11. The biocompatible, therapeutic polymeric nanoparticle of claim 10, wherein the taxane is docetaxel.

12. The biocompatible, therapeutic polymeric nanoparticle of any one of claims 1-9, wherein the therapeutic agent is bortezomib.

13. The biocompatible, therapeutic polymeric nanoparticle of any one of claims 1-12, wherein the nanoparticle further comprises a targeting ligand.

14. A composition comprising a plurality of biocompatible, therapeutic polymeric nanoparticles of any one of claims 1-13, and a pharmaceutically acceptable excipient.

15. A method of treating prostate cancer, breast cancer or multiple myeloma comprising administering to a patient in need thereof an effective amount of any of the therapeutic polymeric nanoparticles of claims 1-13 or the composition of claim 14.

16. A plurality of therapeutic nanoparticles prepared by:
combining a therapeutic agent selected from a taxane and bortezomib, a diblock copolymer of poly(lactic) acid and polyethylene (glycol) or a diblock copolymer of poly(lactic)-co-poly (glycolic) acid-poly(ethylene)glycol, and a glyceride with an organic solvent to form a first organic phase having about 10 to about 40% solids;
combining the first organic phase with a first aqueous solution to form a second phase;
emulsifying the second phase to form an emulsion phase;
quenching the emulsion phase to form a quenched phase;
adding a drug solubilizer to the quenched phase to form a solubilized phase; and
filtering the solubilized phase to recover the nanoparticles, thereby forming a slurry of therapeutic nanoparticles each having about 0.1 to about 35 weight percent of the therapeutic agent.

17. The plurality of therapeutic nanoparticles of claim 16, wherein the glyceride is a monoglyceride comprising lauroyl-rac-glycerol, glycerol monomyristate, glycerol monopalmitate, glycerol monostearate, glycerol monoarachidate, glycerol monobehenate, glycerol monopalmitoleate, glycerol monopalmitoleate, glycerol monooleate, glycerol monolinoleate, glycerol monolinolenate, glycerol monoarachidonate, glycerol monocaprylate, or combinations thereof.

18. The plurality of therapeutic nanoparticles of claim 16 or claim 17, wherein the therapeutic agent is docetaxel.

19. The plurality of therapeutic nanoparticles of claim 16 or claim 17, wherein the therapeutic agent is bortezomib.

20. The plurality of therapeutic nanoparticles of any one of claims 16-19, wherein the glyceride is homogeneously dispersed within the nanoparticle.
